(19) United States
(12) Patent Application Publication
(10) Pub. No.: US 2006/0121547 A1
(43) Pub. Date: Jun. 8, 2006

(54) Diffusion layer for an enzyme-based sensor application

(76) Inventor: Mark McIntire, Alpharetta, GA (US)

Correspondence Address: Roche Diagnostics Corporation, Inc., 9115 Hague Road, PO Box 50457, Indianapolis, IN, 46250-0457, US

(21) Appl. No.: 11/004,210

(22) Filed: Dec. 3, 2004

(51) Int. Cl. C12Q 1/54; C12M 1/34

(52) U.S. Cl. 435/14; 435/287.1

(57) ABSTRACT

A diffusion layer for an enzyme-based sensor application is provided, wherein the diffusion layer comprises (a) at least one polymer material, and (b) particles, typically hydrophilic particles, carrying the enzyme, the hydrophilic particles being dispersed in the at least one polymer material.

### BACKGROUND OF THE INVENTION

The present invention relates to a diffusion layer for an enzyme-based sensor application and to a sensor comprising the same.

Enzyme-based sensors are widely used to determine substances of interest in a qualitative as well as quantitative manner in blood and in other body liquids. Enzyme-based sensors are in particular used for the determination of enzyme substrates. In an enzyme-based sensor a so-called chemical transducer reaction occurs wherein the substance to be determined is converted under participation of at least one enzyme into another substance. Many enzyme-based sensors require participation of a co-substrate. The consumption of the co-substrate or production of the other substance is detected directly or indirectly.

An enzyme-based sensor usually comprises several layers, among them an enzyme layer and a diffusion layer (cover membrane, outer layer). This diffusion layer is in direct contact with the sample and limits the diffusion of the substances necessary for the sensing reaction, especially the enzyme substrate or co-substrate.

Enzyme-based sensors can be provided as electrochemical sensors or as optical sensors (optodes). The construction and function of a glucose optode is for example described in U.S. Pat. No. 6,107,083.

Particularly, enzyme-based sensors which are used for the determination of glucose, lactate or creatinine are preferably constructed with oxidoreductases and the detection is based on the oxygen consumption. In this case, the sensor necessits a cover membrane being a porous or at least a permeable polymer membrane, which controls the permeation of both the enzyme substrate and oxygen.

The glucose sensor using an enzyme is the best known practical measure for detecting saccharides. This technique includes contacting the sample with a sensor, diffusion of glucose into the sensor, decomposition of glucose with the enzyme (glucose oxidase) within an enzymatic layer, and measuring the amount of oxygen consumed by an appropriate means such as a luminescent dye, or, measuring the amount of hydrogen peroxide produced through an appropriate means such as by an amperometric electrode.

Accordingly, enzyme-based sensors can be provided as electrochemical sensors (electrodes) or as optical sensors (optodes). The construction and function of a glucose optode is for example described in U.S. Pat. No. 6,107,083 (Collins et al.). The construction and function of a glucose electrode is for example described in U.S. Pat. No. 6,214,185 (Offenbacher et al.).

Particularly, enzyme-based optodes which are used for the determination of glucose, lactate or creatinine are preferably constructed with oxidoreductases and the detection is predominantly based on the oxygen consumption. The basic design concept of a luminescence-based optode comprises in order

a) a light-transmissive support,

b) an oxygen sensing layer containing a luminescent dye, in a light-transmissive, oxygen permeable matrix,

c) an enzymatic layer containing an oxidoreductase or an enzyme cascade immobilized in a hydrophilic, water and oxygen-permeable matrix,

d) a diffusion layer limiting the diffusion of the enzyme substrate and/or co-substrate into the enzymatic layer, and optionally

e) an optical isolation layer, impermeable to light.

Alternatively, the enzyme layer or the diffusion layer can be constructed from light-impermeable materials in order to function as optical isolation layer.

Prior to sample measurement, the sensor is equilibrated with water or appropriate salt solutions and a certain level of O2, i.e., 150 mm Hg. For measurement, the sensor is contacted with the sample. Glucose diffuses from the sample into the enzymatic layer. The glucose and oxygen consumption within the enzymatic layer results in a depletion of oxygen in the adjacent dye layer. In the case of luminescent dyes, the rate of O2-depletion within the dye layer translates into a corresponding increased luminescence intensity (i.e., expressed as ΔI/Δt). The value of the latter, i.e., determined within a certain time interval after sample contact, is related to the glucose concentration by appropriate correlation functions. In the event that all the O2 is consumed in the dye layer, ΔI/Δt will become zero, as luminescence intensity will not further increase. To account for variations of dye loading (i.e., sensor-to-sensor) or variations in intensity of the light source (instrument-to-instrument) intensity-changes are preferably expressed as ΔI/(IΔt) where I is the intensity at known pO2 (i.e, the intensity measured prior to sample contact). We refer to the latter quantity as slope, where slope is determined in a given time window after sample measurement. Indeed, a number of methods are known to determine the slope. Beside luminescence intensity, luminescence decay-time (i.e., Δτ/Δt), determined by pulse or phase methods known in the art may be used as well.

Selection of the polymer forming the enzymatic layer depends on its a) insolubility in water or the watery sample, b) solubility in solvents not destroying the activity of the enzyme and c) its adhesion properties to the polymer of the adjacent dye layer. A number of non-crosslinked hydrophilic polymers are potential candidate materials. Certain low water uptake polyether-polyurethanes (water content 2.5% in the wet state), soluble in lower alcohols (such as ethanol) or alcohol water mixes are preferred materials to provide good adhesion to dye layers manufactured from certain silicones.

One disadvantage of using very hydrophilic polymers (water content 50% or higher) is that highly water soluble substrates such as glucose and lactate permeate too fast into the enzymatic layer such that the transduction reaction runs too fast, resulting in a too fast (a few seconds or less) depletion of O2 in the dye layer. Aside from a number of other disadvantages, determination of fast rates becomes impractical. The diffusion layer controls the permeation of the enzyme substrate.

According to one approach known in the state of the art, pre-formed cover membranes consisting of non-hydrating micro porous structures from polymers like polycarbonate, polypropylene and polyesters are used to control permeation of the enzyme substrate. The porosity of such membranes is provided by physical means, e.g., by neutron or argon track etching. Glucose permeates across such membranes predominantly through these pores filled with liquid. The co-substrate O2, is filled into the sensor layer prior to contacting the sample. The co-substrate (i.e., O2) permeates through both, the pores and the polymer. The degree of permeation through the polymer depends on its permeability for O2.

One major disadvantage is that pre-formed thin membranes have to be attached to the enzyme layer. Very often the membranes are mechanically attached to the enzyme layer. Mechanical attachment is expensive and technically complex. Further problems occur insofar as it is difficult to apply the membrane onto the underlying layer without producing air bubbles. Similar problems also occur when the membrane is for example glued onto an underlying layer.

Another approach known in the state of the art is to form a diffusion layer by applying a solution of a polymer to the enzyme layer and by evaporating the solvent. Offenbacher et al. (U.S. Pat. No. 6,214,185) describe a cover membrane made of a PVC copolymer which allows a quite satisfying adjustment of the permeability due to the presence of a hydrophilic co-monomer component. Upon exposure to water or aqueous samples, the hydrophilic domains provide a swelled structure acting as a permeation path for the water-soluble enzyme substrate.

### SUMMARY OF THE INVENTION

It is against the above background that the present invention provides certain unobvious advantages and advancements over the prior art. In particular, the inventor has recognized a need for improvements in diffusion layer or membrane design for enzyme-based sensor application.

Although the present invention is not limited to specific advantages or functionality, it is noted that the present invention provides a sensor with a rapid oxygen recovery time, which can also be used for multiple measurements within a short time frame. In addition, a sensor with a short wash time to remove products of the enzymatic reaction is provided, as well as a rapid hydration (“wet-up”) of the enzymatic layer.

In accordance with one embodiment of the present invention, a diffusion layer is provided comprising at least one polymer material, and particles carrying an enzyme. The particles are dispersed in the at least one polymer material. The particles can be hydrophilic.

The invention is based on the idea to combine the diffusion layer and the enzyme layer to one single layer.

In accordance with another embodiment of the present invention, the diffusion layer can further comprise particles for optical isolation, e.g., particles dispersed in the at least one polymeric material.

In accordance with still another embodiment of the present invention, an enzyme-based sensor is provided comprising the diffusion layer according to the invention, which can be the cover layer of the sensor.

In accordance with yet another embodiment of the present invention, an enzyme-based sensor is provided comprising at least one dye layer.

In accordance with yet still another embodiment of the present invention, the sensor is an electrochemical sensor or an optical sensor.

Another aspect of the present invention is the use of the enzyme-based sensor for the detection and/or qualitative and/or quantitative determination of an enzyme substrate, in particular glucose, and/or co-substrate. The inventive enzyme-based sensor can be used in blood, wherein typically multiple measurements are performed.

In accordance with yet still another embodiment of the present invention, a method of preparing a diffusion layer for an enzyme-based sensor is provided comprising (i) forming a dispersion comprising at least one polymer material and enzyme-carrying particles; (ii) applying the dispersion directly on an underlying layer to form an enzyme-carrying diffusion layer; and (iii) drying the dispersion.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 is a schematic illustration of an optical measuring system shown in accordance with one embodiment of the present invention;

FIG. 2 shows the oxygen recovery time of a state of the art glucose sensor;

FIG. 3 shows luminescence intensity versus oxygen recovery time (sec) of a glucose sensor according to one embodiment of the present invention;

FIG. 4 shows the kinetic luminescence intensity response curves of the sensor according to FIG. 3;

FIG. 5 is a comparison of the calculated glucose concentration in whole blood, calculated from the measured luminescence intensity; and

FIG. 6 is a comparison of the calculated slopes determined from sensors according to one embodiment of the present invention (enzyme layer mixtures B, C, D, E) using whole blood, and gravimetric glucose standards containing 30, 70, 150, 300 and 400 mg/dl glucose, respectively.

Skilled artisans appreciate that elements in the figures are illustrated for simplicity and clarity and have not necessarily been drawn to scale. For example, the dimensions of some of the elements in the figures may be exaggerated relative to other elements to help improve understanding of the embodiment(s) of the present invention.

### DETAILED DESCRIPTION OF TYPICAL EMBODIMENTS OF THE INVENTION

In accordance with one embodiment of the present invention, a diffusion layer is provided comprising enzyme-carrying particles and optionally particles dispersed in at least one polymeric material. The particles can be hydrophilic. The permeability of the layer for the co-substrate may be provided by the swelled structure of the at least one polymer acting as an adjustable permeation path for the water-soluble enzyme substrate and the swelled structure of the enzyme-carrying particle.

The polymer material used for the layer of the present invention can generally be any polymer material or a mixture of polymer materials with adjustable swelled structure, soluble in non-enzyme destroying, non-toxic, typically easily volatile and easily applicable solvents or mixture of solvents.

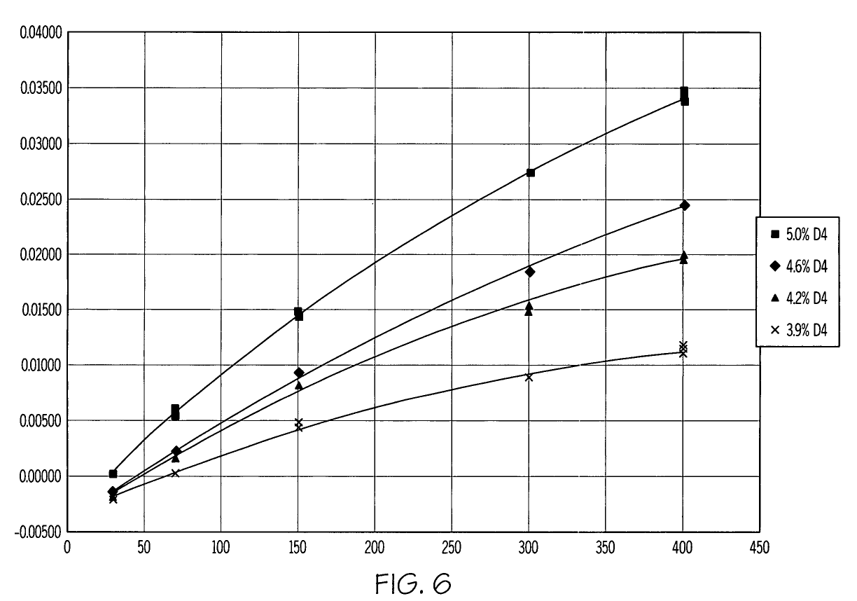

In accordance with the present invention, it is also possible to add to the polymer up to 20% by weight high water uptake polyether-polyurethane co-polymers (water content 50% in the wet state). Such addition results in a polymer mix with adjustable water content. The advantage is an adjustable slope (compare FIG. 6). The enzyme may be incorporated in such layers, for example, immobilized to hydrophilic particles or suspended in the polymer forming the enzymatic layer.

Typical polymer materials can be selected from the group consisting of non-crosslinked, non-water soluble polymers and more typically from low-water uptake (<40%, typically <20% by weight) polyether-polyurethane co-polymers.

Due to the various selection possibilities with regard to the polymer material, a layer of the invention can be provided easily, which can be applied directly by way of a solution. The layer can for example be coated on an underlying layer, typically onto an oxygen sensitive layer of an oxygen optode. It is an advantage of the layer of the present invention that a combined diffusion-enzyme layer can easily be provided, which is insoluble in the sample to be measured (i.e, in body liquids such as serum, plasma and blood).

The combined diffusion-enzyme layer according to the present invention comprises hydrophilic enzyme-carrying particles dispersed in the layer forming polymer material. Both the particles and the polymer provide the permeability for the co-substrate and thus the fast oxygen recovery of the sensor.

The enzymatic layer has a defined permeability to the enzyme substrate, which is provided by the density of the substrate-permeable particles, formed by the size and amount of particles according to the present invention. According to the application of the layer, the size and amount of the particles can be varied.

For the use as particles in the membrane, essentially all stable hydrophilic particles and mixtures of such particles are useful, which possess an inherent and defined water uptake and enzyme loading. According to the desired application and/or water-uptake and enzyme loading, suitable particles can be elected.

Examples for suitable particles include gel particles. Typical particles are based on polyacrylamide, polyacrylamide and N-acryloxysuccinimide copolymers, polyvinylpyrrolidone, polyvinylacetate, and agarose beads. It is contemplated that essentially all stable non-hydrophilic particles with surface-bound enzyme and mixtures of such particles may also be useful. Examples for such particles include glass, quartz, cellulose, polystyrene, nylon and other polyamides.

The enzymatic layer according to the present invention can further comprise other elements such as carbon black and titanium dioxide for optical isolation and for improved remission properties of an optical sensor.

The thickness of the enzymatic layer according to the invention can be chosen flexibly with regard to the desired use. Thickness depends on the size of the enzyme-carrying particles. Suitable thicknesses are within the range of about 1 to about 100 μm, typically about 1 to about 50 μm, more typically about 1 to about 20 μm.

In one embodiment of the enzymatic layer according to the present invention, the size of the particles corresponds at least to the thickness of the layer. In another embodiment, the size of the particles is chosen in a way that the size of single particles or clusters of single particles is smaller then the thickness of the layer.

The enzyme-based sensor of the present invention can further comprises at least one underlaying dye layer or a base electrode. Depending on the type of the sensor, further layers can for example be an interference-blocking layer, a layer for optical isolation, an electro-conductive layer, or a base electrode.

Since the permeability of the diffusion layer can be adjusted as desired, the enzymatic layer provides a fast regeneration of the sensor. In the case of a sensing reaction based for example on the consumption of oxygen, the oxygen permeation can be adjusted in such a manner that the sensor regeneration, e.g., the regeneration of the oxygen reservoir is very fast. Thus, the sensor of the present invention can also be used for multiple measurements.

The enzyme layer of the enzyme-based sensor can for example comprise oxidative enzymes as for example glucose oxidase, cholesterol oxidase or lactate oxidase. The enzyme layer may also comprise an enzyme mixture, such as an enzyme cascade, which makes possible the detection of analytes which cannot be directly detected (via one enzyme reaction), such as the creatine. Creatine cannot be enzymatically oxidized by a simple enzyme but requires several enzymatic steps to generate an analyte derivative, which is detectable by optical or amperometric means. A suitable enzyme cascade system for the detection and/or determination of creatinin comprises, e.g., creatinine amidohydrase, creatinine amidohydrolase, and Sarcosine oxidase.

In the sensor according to one embodiment of the present invention, the enzymatic layer is typically deposited as a cover layer. In this case, after solvent evaporation of the dispersion a stable cover layer is formed. The enzymatic layer is further typically coated directly on an underlying layer, typically a dye layer or an electrode. By a direct coating of the enzymatic layer, typically, the enzymatic layer is attached to the underlying layer by physical adhesion without mechanical fixation and/or use of glue layer.

The enzyme-based sensor of the present invention can represent any kind of a biosensor. Examples for suitable biosensors are, for example, optical sensors. With typical optical sensors, the consumption of oxygen due to an enzymatic reaction can be detected using an appropriate dye which is sensitive to oxygen, e.g., a luminescent dye quenchable by oxygen.

Suitable dyes for use in the sensor of the present invention are selected from the group consisting of ruthenium(II), osmium(II), iridium(III), rhodium(III) and chromium(III) ions complexed with 2,2′-bipyridine, 1,10-phenanthroline, 4,7-diphenyl-1,10-phenanthroline, 4,7-dimethyl-1,10-phenanthroline, 4,7-disulfonated-diphenyl-1,10-phenanthroline, 5-bromo-1,10-phenanthroline, 5-chloro-1,10-phenathroline, 2,2′-bi-2-thiazoline, 2,2′-dithiazole, VO²⁺, Cu²⁺, Zn²⁺, Pt²⁺, and Pd²⁺ complexed with porphyrin, chlorine and phthalocyanine, and mixtures thereof. In a typical embodiment, the luminescent dye is [Ru(diphenylphenantroline)₃], octaethyl-Pt-porphyrin, octaethyl-Pt-porphyrin ketone, or tetrabenzo-Pt-porphyrin.

Furthermore, an electrochemical sensor is suitable for the use in the present invention.

A further aspect of the present invention is the use of an enzyme-based sensor as described above for the detection or quantitative determination of a substance, typically an enzyme substrate.

In the field of medicine, a possibility of the use is for example the determination of physiological parameters. A determination and/or detection can be carried out in any liquid, for example in various body liquids such as blood, serum, plasma, urine, and the like. A typical use of the sensor is a detection and/or determination of analytes in blood.

A possible use of the sensors according to the invention is for example the determination of blood glucose in patients suffering from diabetes. Other metabolic products that can be determined with the enzyme-based sensor according to the invention are for example cholesterol or urea.

Another possible use of the enzyme-based sensor of the invention is in the field of environmental analytics, process control in biotechnology, and food control.

With the use according to the invention of the enzyme-based sensor a wide variety of substances, for example enzyme substrates and/or co-substrates can be determined and/or detected. Suitable enzyme substrates are for example cholesterol, sucrose, glutamate, ethanole, ascorbic acid, fructose, pyruvat, glucose, lactate or creatinine. Typically, a determination and/or detection of glucose, lactate or creatinine is performed. A more typical substance to be detected and/or determined is glucose.

Since the regeneration of the enzyme-based sensor can be influenced by adjusting the permeation, the regeneration is fast enough to allow multiple measurements. In a typical use of the sensor multiple measurements are performed. Further, the enzyme-based sensor can be employed for every sensor-application known in the art, such as for a single use application for multi-use applications.

In accordance with yet another embodiment of the present invention, a method for the preparation of a diffusion layer for an enzyme-based sensor as described above is provided. This method comprises:

(i) forming a dispersion comprising
(a) at least one polymer material, and
(b) enzyme-carrying particles, typically of hydrophilic nature,

(ii) applying the dispersion directly on an underlying layer to form an enzyme-carrying diffusion layer; and

(iii) drying the dispersion.

The method according to the invention allows a direct casting of the layer due to the broad option of polymer materials. Further, the materials can be elected in a way that heating of the dispersion is not necessary. Thus, by the method according to the invention, an easy handling is provided.

In the method according to the invention, the dispersion is typically attached to the underlaying layer by physical adhesion. Also, drying the dispersion can comprise removing a solvent from the dispersion.

In order that the invention may be more readily understood, reference is made to the following examples, which are intended to illustrate the invention, but not limit the scope thereof.

### EXAMPLES

### Example 1

### Preparation of Oxygen Dye Particles

| Material | Concentration | |
|---|---|---|
| Tris(1,10-phenanthrpline)ruthenium(II) | 61.5 | grams |
| chloride hydrate (cat. 34,371-4) | | |
| Aldrich Chemical Co., Inc., 1001 West Saint | | |
| Paul Ave., Milwaukee, WI 53233 | | |
| 100 mM Phosphate buffer pH 7.5 | 7.5 | grams |
| Silica Gel (cat. 4115-100) | 2.25 | grams |
| Whatman Inc., 9 Bridewell Place, | | |
| Clifton, NJ 07014 | | |

The dye tris-(1,10-phenanthroline) Ru (II) chloride was adsorbed onto silicagel particles according to a procedure published in: O. S. Wolfbeis, M. J. P. Leiner and H. E. Posch, “A new sensing material for optical oxygen measurement with the indicator embedded in an aqueous phase”, Microchim. Acta, III (1986) 359.

### Example 2

### Preparation of the Oxygen Layer Mixture

| Material | Concentration | |
|---|---|---|
| O₂ Ruthenium-silica dye particles | 0.5 | grams |
| Pressure Sensitive Adhesive (cat. PSA590) | 4 | grams |
| GE Silicones, 260 Hudson River Road, | | |
| Waterford, NY 12188 | | |
| Toluene | 2 | grams |
| Aldrich Chemical Co., Inc., 1001 West Saint | | |
| Paul Ave., Milwaukee, WI 53233 | | |

Add the Toluene to the Pressure Sensitive Adhesive and mix until homogeneous. Add this solution to the O₂ indicator dye and mix for 16 hours.

### Example 3

### Preparation of Enzyme-Carrying Hydrophilic Particles

TABLE 1
Glucose Oxidase Immobilization

| Material | Concentration | |
|---|---|---|
| CarboLink Coupling Gel (cat. 20391ZZ) | 5 | grams |
| Pierce, 3747 North Meridian Road, | | |
| Rockford, IL 61105 | | |
| Glucose Oxidase (cat. 1939998) | 0.15 | grams |
| Roche Molecular Biochemicals, 9115 Hague | | |
| Road, Indianapolis, IN 46250 | | |
| Sodium Periodate | 0.015 | grams |
| Aldrich Chemical Co., Inc., 1001 West Saint | | |
| Paul Ave., Milwaukee, WI 53233 | | |
| 100 mM Phosphate buffer pH 7.5 | 15 | mL |
| D-Salt Polyacrylamide Plastic Desalting | 10 | mL column |
| column (cat. 43243ZZ) | | |
| Pierce, 3747 North Meridian Road, | | |
| Rockford, IL 61105 | | |

The Sodium Periodate was added to 5 mL of 100 mM phosphate buffer and stirred for 10 minutes. To this solution was added the glucose oxidase, this solution was stirred at room temperature for 30 minutes. The solution was pippetted and added to the pre-filled polyacrylamide desalting column. The desalted glucose oxidase was collected in an appropriate container. The column was washed with 10 mL of 100 mM phosphate buffer to wash out the remaining glucose oxidase. The glucose oxidase was then added to 5 grams of the CarboLink Coupling gel and incubated, with gentle mixing, at room temperature for 24 hours. The glucose oxidase-agarose beads were then added to 10 mL of 100 mM phosphate buffer. The solution was centrifuged and the top layer decanted off.

### Example 4

### Enzyme Layer Mixture A

| Material | Concentration | |
|---|---|---|
| Polyurethane type 138-03 lot #RL151-87 | 3 | grams |
| polymer | | |
| Tyndale Plains-Hunter Ltd., 17K Princess | | |
| Road, Lawerenceville, NJ 08551 | | |
| Carbon Black (cat. 1810) | 0.3 | grams |
| Degussa Corp./William B. Tabler Co., | | |
| Ormsby Place Industrial Park, 1331 S. 15^{th} | | |
| St., Louisville, KY 40210 | | |
| Absolute Ethanol (200 Proof) | 6.7 | grams |
| Aldrich Chemical Co., Inc., 1001 West Saint | | |
| Paul Ave., Milwaukee, WI 53233 | | |
| Glucose Oxidase coupled to CarboLink | 5 | grams |
| Coupling Gel (Example 3) | | |

Ethanol was added to the polyurethane and mixed until dissolved. The carbon black was added to this solution and mixed 24 hours. To this solution was added the glucose oxidase-agarose beads and mixed until homogenous.

### Example 5

### Enzyme Layer Mixture B

| Material | Concentration | |
|---|---|---|
| Polyurethane type 138-03 lot #RL151-87 | 2.1 | grams |
| polymer | | |
| Tyndale Plains-Hunter Ltd., 17K Princess | | |
| Road, Lawerenceville, NJ 08551 | | |
| Polyurethane type D4 lot #140-42 polymer | 0.3 | grams |
| Tyndale Plains-Hunter Ltd., 17K Princess | | |
| Road, Lawerenceville, NJ 08551 | | |
| Carbon Black (cat. 1810) | 0.3 | grams |
| Degussa Corp./William B. Tabler Co., | | |
| Ormsby Place Industrial Park, 1331 S. 15^{th} | | |
| St., Louisville, KY 40210 | | |
| Absolute Ethanol (200 Proof) | 7.3 | grams |
| Aldrich Chemical Co., Inc., 1001 West Saint | | |
| Paul Ave., Milwaukee, WI 53233 | | |
| Glucose Oxidase coupled to CarboLink | 5 | grams |
| Coupling Gel | | |

Ethanol was added to the type 138-03 polyurethane and mixed until dissolved. Polyurethane type D4 was added next to the solution and mixed until dissolved. The carbon black was added to this solution and mixed for 24 hours. To this solution was added the glucose oxidase-agarose beads and mixed until homogenous.

### Example 6

### Enzyme Layer Mixture C

| Material | Concentration | |
|---|---|---|
| Polyurethane type 138-03 polymer | 2.025 | grams |
| Tyndale Plains-Hunter Ltd., 17K Princess | | |
| Road, Lawerenceville, NJ 08551 | | |
| Polyurethane type D4 lot #140-42 polymer | 0.325 | grams |
| Tyndale Plains-Hunter Ltd., 17K Princess | | |
| Road, Lawerenceville, NJ 08551 | | |
| Carbon Black (cat. 1810) | 0.3 | grams |
| Degussa Corp./William B. Tabler Co., | | |
| Ormsby Place Industrial Park, 1331 S. 15^{th} | | |
| St., Louisville, KY 40210 | | |
| Absolute Ethanol (200 Proof) | 7.35 | grams |
| Aldrich Chemical Co., Inc., 1001 West Saint | | |
| Paul Ave., Milwaukee, WI 53233 | | |
| Glucose Oxidase coupled to CarboLink | 5 | grams |
| Coupling Gel | | |

Ethanol was added to the type 138-03 polyurethane and mixed until dissolved. Polyurethane type D4 was added next to the solution and mixed until dissolved. The carbon black was added to this solution and mixed for 24 hours. To this solution was added the glucose oxidase-agarose beads and mixed until homogenous.

### Example 7

### Enzyme Layer Mixture D

| Material | Concentration | |
|---|---|---|
| Polyurethane type 138-03 polymer | 1.95 | grams |
| Tyndale Plains-Hunter Ltd., 17K Princess | | |
| Road, Lawerenceville, NJ 08551 | | |
| Polyurethane type D4 lot polymer | 0.35 | grams |
| Tyndale Plains-Hunter Ltd., 17K Princess | | |
| Road, Lawerenceville, NJ 08551 | | |
| Carbon Black (cat. 1810) | 0.3 | grams |
| Degussa Corp./William B. Tabler Co., | | |
| Ormsby Place Industrial Park, 1331 S. 15^{th} | | |
| St., Louisville, KY 40210 | | |
| Absolute Ethanol (200 Proof) | 7.4 | grams |
| Aldrich Chemical Co., Inc., 1001 West Saint | | |
| Paul Ave., Milwaukee, WI 53233 | | |
| Glucose Oxidase coupled to CarboLink | 5 | grams |
| Coupling Gel | | |

Ethanol was added to the type 138-03 polyurethane and mixed until dissolved. Polyurethane type D4 was added next to the solution and mixed until dissolved. The carbon black was added to this solution and mixed for 24 hours. To this solution was added the glucose oxidase-agarose beads and mixed until homogenous.

### Example 8

### Enzyme Layer Mixture E

TABLE 2

| Material | Concentration | |
|---|---|---|
| Polyurethane type 138-03 polymer | 11.875 | grams |
| Tyndale Plains-Hunter Ltd., 17K Princess | | |
| Road, Lawerenceville, NJ 08551 | | |
| Polyurethane type D4 polymer | 0.375 | grams |
| Tyndale Plains-Hunter Ltd., 17K Princess | | |
| Road, Lawerenceville, NJ 08551 | | |
| Carbon Black (cat. 1810) | 0.3 | grams |
| Degussa Corp./William B. Tabler Co., | | |
| Ormsby Place Industrial Park, 1331 S. 15^{th} | | |
| St., Louisville, KY 40210 | | |
| Absolute Ethanol (200 Proof) | 7.45 | grams |
| Aldrich Chemical Co., Inc., 1001 West Saint | | |
| Paul Ave., Milwaukee, WI 53233 | | |
| Glucose Oxidase coupled to CarboLink | 5 | grams |
| Coupling Gel | | |

Ethanol was added to the type 138-03 polyurethane and mixed until dissolved. Polyurethane type D4 was added next to the solution and mixed until dissolved. The carbon black was added to this solution and mixed for 24 hours. To this solution was added the glucose oxidase-agarose beads and mixed until homogenous.

### Example 9

### Construction of the O2-Sensitive Layer

The silicone adhesive containing the oxygen sensitive fluorescent dye (Example 2) was knife coated (knife high setting 120 um) on top of a 126 urn Melinex 505 polyester substrate. The oxygen sensitive layer was dried to 33 um thickness.

### Example 10

### Construction of the Enzymatic Layer

For construction of the enzyme layer, mixtures A,B,C,D and E, respectively were knife coated (knife high setting 200 um) on top of the dry oxygen sensitive layer (Example 9). After 1 hour the enzyme layer measured 38 um.

### Example 11

General methods of preparation, cutting and measuring sensor disks were described by Trettnak et al. in Analyst, 113 (1988) 1519-1523 (“Optical sensors”); Moreno-Bondi et al. in Anal. Chem., 62 (1990) 2377-2380 (“Oxygen optode for use in a fiber-optic glucose biosensor”); M. J. P. Leiner and P. Hartmann in Sensors and Actuators B, 11 (1993) 281-289 (“Theory and practice in optical pH sensing”).

From the individual foils (Example 10) sensor disks of the invention were punched out and used in a gas-tight flow-through chamber heated to 37° C., comprising a transparent wall, a channel, an inlet and an outlet opening for introduction of gases and solutions (not illustrated).

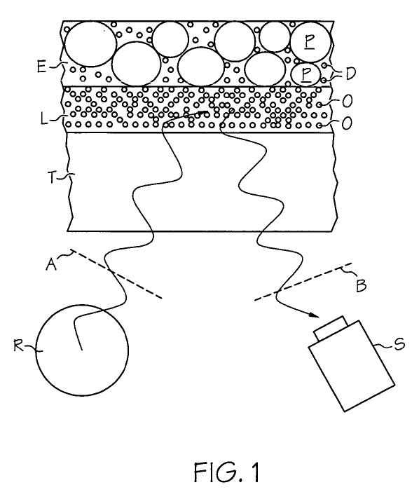

The experimental results can be seen with the attached FIGS. 1-6.

FIG. 1 shows an illustration of the optical measuring system according to a typical embodiment of the invention. R denotes a blue LED as light source, S a photodiode as detector, A and B optical filters for selecting the excitation and emission wavelengths receptively, an optic arrangement for conducting the excitation light into the dye layer L and the emission light to the photodetector S as well as a device for electronic signal processing (not illustrated). At the excitation end an interference filter A (peak transmission at 480 nm) and at the emission end a 520 nm cut-off filter B was used. E denotes the emzyme layer comprising enzyme carrying particles P and D (black carbon). L denotes the dye layer, O the oxygen sensitive dye and T the light transmissive support.

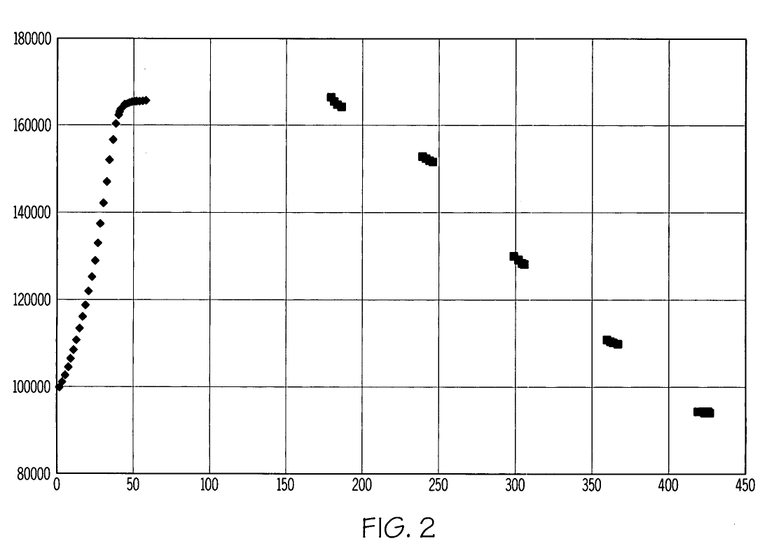

FIG. 2 shows the oxygen recovery time of a state of the art glucose sensor. An aqueous sample was introduced into the measuring chamber containing a state of the art optical glucose sensor, which uses a RoTrac-capillary pore membrane attached on top of the enzymatic layer to control the glucose and oxygen diffusion into the sensor. The enzymatic layer consists of a hydrophilic polymer containing hydrophilic agarose beads with immobilised enzyme (glucose oxidase). Prior measurement the enzyme layer was activated (hydrated) with water and equilibrated with gas containing 100 mmHg O2 partial pressure (not shown). The sample containing 200 mg/dl glucose was introduced into the cell and the fluorescence was measured for 60 seconds. The enzyme glucose oxidase in the enzyme layer converted the glucose from the sample to gluconolactone, thereby consuming oxygen as a co-substrate. Consumption of O2 results in a depletion of the oxygen contained in the adjacent dye layer. The O2 sensitive luminescent dye present in the dye layer responds with increasing luminescence intensity. The glucose sensor was then washed with a pH 7.4 buffer solution for 2 minutes to remove unconsumed glucose. Then gas containing 90 mmHg oxygen was pumped across the cell and the luminescence intensity returned back to the intensity level as initially (corresponding to 100 mmHg O2). FIG. 2 shows the measured luminescence intensity versus time (sec). The oxygen recovery time was greater than 4 minutes.

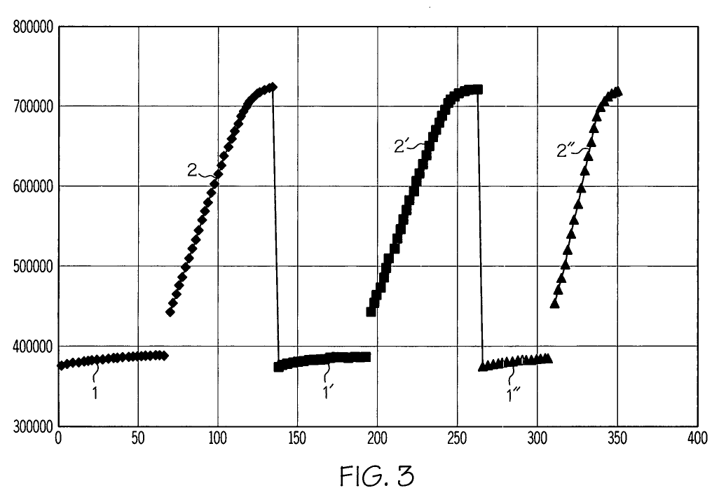

FIG. 3 shows luminescence intensity versus oxygen recovery time (sec) of a glucose sensor according to the invention. The sensor was prepared according Examples 9 and 10, using enzyme layer mixture A. Base line 1 denotes the luminescence according to the initial O2 content.

Then a sample containing 200 mg/dL glucose was introduced to the glucose sensor of the invention. Luminescence intensity was measured for 60 seconds and increased according to line 2; the enzyme (glucose oxidase) in the sensor converted the glucose contained in the sample to gluconolactone, consuming oxygen and thereby depleting the oxygen reservoir in the sensor leading to the increase in luminescence intensity.

Then the glucose sensor was washed with a pH 7.4 buffer for 2 minutes to remove unconsumed glucose. 100 torr oxygen was pumped across the sensor and monitored until the oxygen fluorescent intensity returned to the same fluorescent intensity as initially (line 1′). This procedure was repeated twice to look at oxygen recovery consistency (lines 2′; 1″ and 2″). The inventive glucose sensor exhibited an oxygen recovery time which was less than the wash time of 120 seconds.

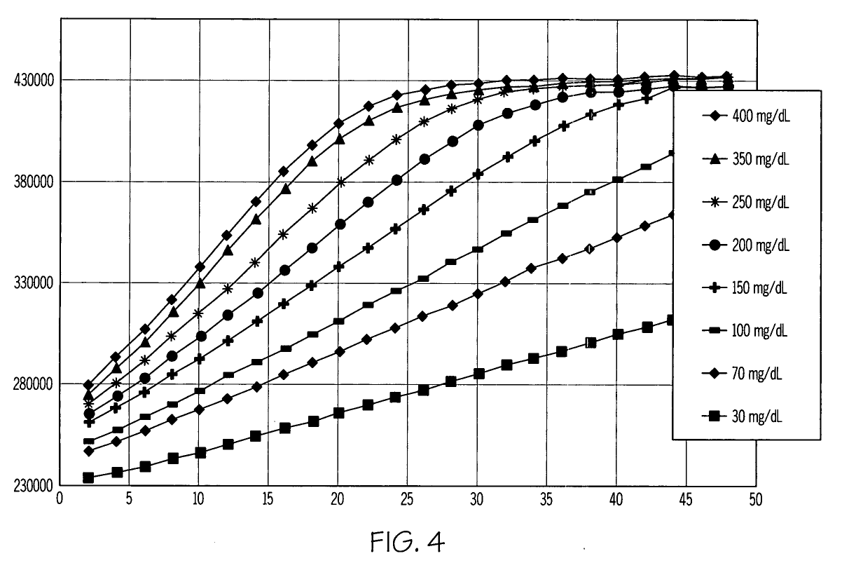

FIG. 4 shows the kinetic luminescence intensity response curves of the sensor according FIG. 3 for aqueous samples ranging from 30 to 400 mg/dL glucose using the glucose sensor according to the invention.

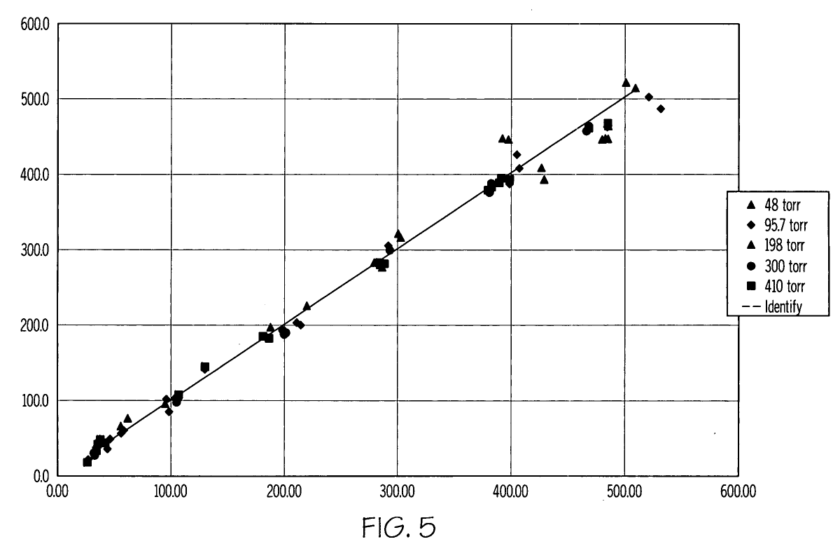

FIG. 5 is a comparison of the calculated glucose concentration in whole blood, calculated from the measured luminescence intensity. The chart shows good agreement between a reference instrument and the glucose sensor according to the invention (R²=0.9949).

FIG. 6 is a comparison of the calculated slopes determined from sensors according to the invention (enzyme layer mixtures B, C, D, E) using whole blood gravimetric glucose standards, containing 30, 70, 150, 300 and 400 mg/dl glucose, respectively.

As can be seen from FIG. 6, the higher the water content of the enzyme layer forming polymer, the higher the slopes—under otherwise essentially same conditions (total amount of polymer and particles). A further increase of the water content would yield even higher slopes. With respect to a given selected time window (7-13 seconds after sample contact) there is a limitation for allowable maximum slope.

For determination of slopes the luminescence intensity I_{cal} of the sensor equilibrated with 90 mm Hg was measured prior contacting the sensor with sample. Then the sample was introduced and four intensities I₁, I₂, I_{3, I}₄ at t₁=7, t₂=9, t₃=11, t₄=13 seconds after sample contact were recorded. To account for variation of dye loading (sensor-to-sensor) the 4 intensities were then each divided by I_{cal} to yield for intensities I_{1c}, I_{2c}, I_{3c}, I_{4c}. With the data pairs (t₁, I₁; t₂ I₂; t₃ I₃; t₄ I₄ ) a linear regression was performed according to the equation y=a+bx where b denotes the slope.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A diffusion layer comprising:
at least one polymer material, and
particles carrying an enzyme, wherein said particles are dispersed in said at least one polymer material.

2. The diffusion layer of claim 1, wherein said particles are hydrophilic.

3. The diffusion layer of claim 1 further comprising particles for optical isolation, wherein said particles for optical isolation are dispersed in said at least one polymer material.

4. The diffusion layer of claim 1, wherein said diffusion layer has a thickness between about 1 and about 100 μm.

5. The diffusion layer of claim 1, wherein said diffusion layer has a thickness between about 1 and about 50 μm.

6. The diffusion layer of claim 1, wherein said diffusion layer has a thickness between about 1 and about 20 μm.

7. An enzyme-based sensor comprising a diffusion layer according to claim 1.

8. The enzyme-based sensor of claim 7 comprising at least one dye layer.

9. The enzyme-based sensor of claim 7, wherein said diffusion layer according to claim 1 is the cover layer.

10. The enzyme-based sensor of claim 7, wherein said sensor is an electrochemical sensor or an optical sensor.

11. Use of an enzyme-based sensor according to claim 7 for the detection and/or qualitative and/or quantitative determination of an enzyme substrate and/or co-substrate.

12. Use according to claim 11, wherein said enzyme substrate is glucose.

13. Use according to claim 12, wherein said determination is performed in blood.

14. Use according to claim 11, wherein multiple measurements are performed.

15. A method of preparing a diffusion layer for an enzyme-based sensor comprising:
(i) forming a dispersion comprising at least one polymer material and enzyme-carrying partricles;
(ii) applying said dispersion directly on an underlying layer to form an enzyme-carrying diffusion layer; and
(iii) drying the dispersion.

16. The method of claim 15, wherein the drying comprises removing a solvent from the dispersion.
